(19) United States
(12) Patent Application Publication
(10) Pub. No.: US 2009/0047366 A1
(43) Pub. Date: Feb. 19, 2009

(54) Inorganic Coagulation Accelerators for Individuals taking Platelet Blockers or Anticoagulants

(76) Inventors: Robert L. Bedard, McHenry, IL (US); Carl N. Zenz, Muskego, WI (US)

Correspondence Address: HONEYWELL INTERNATIONAL INC;PATENT SERVICES, 101 COLUMBIA DRIVE, P O BOX 2245 MAIL STOP AB/2B, MORRISTOWN, NJ, 07962, US

(21) Appl. No.: 11/839,041

(22) Filed: Aug. 15, 2007

(51) Int. Cl. A61K 33/06; A61K 33/00; A61K 47/00; A61K 9/00; A61P 7/00

(52) U.S. Cl. 424/684; 424/400; 424/402; 424/600; 424/682; 514/789

(57) ABSTRACT

The present invention is a method to accelerate the coagulation of blood through the application of inorganic materials to the wound of a patient on anticoagulant or platelet blocker therapy. The method comprises contacting such wounds with a substance selected from the group consisting of zeolitic molecular sieves and non-zeolitic molecular sieves, diatomaceous earth, glass powder or fibers, precipitated or fumed silica, kaolin and montmorillonite clays and Ca exchanged permutites.

### BACKGROUND OF THE INVENTION

The present invention relates to blood clotting agents/medical devices and methods of controlling bleeding in patients who are on medications that result in a suppressed coagulation system.

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some instances, the wound and the bleeding are minor, and normal blood clotting functions without significant outside aid in stopping the bleeding. Unfortunately, in other circumstances, substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner. Moreover, severe wounds can be inflicted in very remote areas or in situations, such as on a battlefield, where adequate medical assistance is not immediately available. In these instances, it is important to stop bleeding, even in less severe wounds, long enough to allow the injured person or animal to receive medical attention. In addition, it may be desirable to accelerate the clotting of even minor wounds to allow the injured person to resume their normal activities.

In an effort to address the above-described problems, materials have been developed for controlling excessive bleeding in situations where conventional aid is unavailable or less than optimally effective. Although these materials have been shown to be somewhat successful, they are not effective enough for traumatic wounds and tend to be expensive. Furthermore, these materials are sometimes ineffective in all situations and can be difficult to apply as well as remove from a wound. Additionally, or alternatively, some materials, especially those of organic origin, can produce undesirable side effects.

Compositions for promoting the formation of clots in blood have also been developed. Such compositions include those that contain zeolites and binders. The use of activated zeolites was disclosed by Hursey et al. in U.S. Pat. No. 4,822,349. It was recognized that the use of these activated zeolites in the clotting of blood generated heat and Hursey et al. stated that the heat was important in achieving a cauterization effect as well as increasing coagulation of the blood. In US 2005/0074505 A1, there is described the use of a zeolite that is exchanged with calcium ions to a very high level. Currently clay-bound Ca-exchanged zeolite A is being sold in an activated form by Z-Medica as a hemostatic treatment for hemorrhages. On some occasions, this calcium exchanged zeolite A has been reported to exhibit an undesirable exothermic effect upon use.

Blood clot formation is a complex process. Several principles are useful in understanding coagulation. In general, the clotting proteins circulate normally as inactive precursors. Coagulation involves a series of activation reactions that in turn act as the catalysts for the next level of reactions and hence, the frequent term “coagulation cascade”. During the reaction(s) process, these proteins and the fibrin mass itself, is highly unstable and water-soluble. This unstable condition will continue until the very final aspects of coagulation. In addition, without (or in limited quantities) those clotting proteins (or in the presence of anticoagulants, i.e., heparin), clotting becomes delayed or prolonged. Eventually, however, fibrin (the foundation of a blood clot) will be formed. This occurs with the cleaving of fibrinogen, one of the coagulation proteins. Finally, Factor XIII (stabilizing factor) is activated by thrombin to yield cross-linked fibrin, which is highly insoluble and stable in formation.

There are several medications that can are commonly prescribed to patients that can result in a lengthened clotting time. Aspirin is a common medication that is well known to interfere with the clotting mechanism to some degree. Warfarin is prescribed to people with an increased tendency for thrombosis or as a prophylaxis in those individuals who have already formed a blood clot (thrombus) which required treatment. This can help prevent formation of future blood clots and help reduce the risk of embolism (migration of a thrombus to a spot where it blocks blood supply to a vital organ). The only common side-effect of warfarin is hemorrhage (bleeding). The risk of severe bleeding is small but definite (1-2% annually). Antiplatelet drugs such as Clopidogrel is another medication prescribed for similar patients. It is a potent oral antiplatelet agent often used in the treatment of coronary artery disease, peripheral vascular disease, and cerebrovascular disease. Clopidogrel is marketed by Bristol-Myers Squibb and Sanofi-Aventis under the trade name Plavix. Due to the difficulty in controlling bleeding in patients who are taking these medications, it is even more important to develop products to treat bleeding in such patients. Now with the present invention, such products and associated methods of treatment of bleeding have been developed.

### SUMMARY OF THE INVENTION

It has been found that many inorganic materials will accelerate the coagulation of blood. Included in these inorganic materials are zeolites, especially calcium exchanged zeolites. In addition, it has been found that solids that can be used to activate the coagulation of platelet-poor plasma in the APTT clinical test or whole blood in the ACT clinical test will also serve as a coagulation accelerator in vivo. In addition, a variety of other materials have been found that can also accelerate blood clotting. Typical materials that can be used for in-vivo clotting include diatomaceous earth, glass powder or fibers, precipitated or fumed silica, kaolin and montmorillonite clays, Ca exchanged permutites. These materials can be used in an aqueous slurry, dry powder or dehydrated forms, and can also be bound with suitable organic or inorganic binders. Surprisingly, these materials exhibit a significant effective level of clotting even in patients on anticoagulation or platelet blocker therapy that is comparable to the effect of such materials on patients not undergoing such therapy.

### DETAILED DESCRIPTION OF THE INVENTION

Patients on anticoagulant and platelet blocker therapy are at risk of haemorrhage because of a suppressed coagulation system. Inorganic coagulation accelerators have been found that counter the effect of anticoagulants and platelet blocker therapy to the extent that such blood clotting time is reduced to a time comparable to patients who are not undergoing such therapy.

Non-limiting examples of these inorganic coagulation accelerators include zeolitic molecular sieves and non-zeolitic molecular sieves. Zeolites are crystalline aluminosilicate compositions which are microporous and which are have a three-dimensional oxide framework formed from corner sharing AlO₂ and SiO₂ tetrahedra. Both naturally occurring and synthetic zeolites can be used. Non limiting examples of zeolites which can be used are the family of zeolites of structure type X, Y, A, beta, etc. Included in these zeolites are the as synthesized zeolites and those that have been exchanged with other cations, e.g. Ca. Non-zeolite molecular sieves are those which do not contain both Al₂O₃ and SiO₂ tetrahedra as essential framework constituents, but which exhibit the ion-exchange and/or adsorption characteristics of the zeolites. In addition, such inorganic materials as diatomaceous earth, glass powder or fibers, precipitated or fumed silica, kaolin and montmorillonite clays and Ca exchanged permutites have been found to be effective.

Diatomaceous earth is a naturally occurring, soft, chalk-like sedimentary rock that is easily crumbled into a fine white to off-white powder. This powder has an abrasive feel, similar to pumice powder and is very light, due to its high porosity. It is composed primarily of silica and consists of fossilized remains of diatoms, a type of hard-shelled algae.

Bioactive glasses are a group of surface reactive glass-ceramics and include the original bioactive glass, Bioglass®. The biocompatibility of these glasses has led them to be investigated extensively for use as implant materials in the human body to repair and replace diseased or damaged bone.

The apparatus that was used to measure the effectiveness of various materials for blood clotting was a TEG® analyzer from Haemoscope Corp. of Morton Grove, Ill. This apparatus measures the time until initial fibrin formation, the kinetics of the initial fibrin clot to reach maximum strength and the ultimate strength and stability of the fibrin clot and therefore its ability to do the work of hemostasis—to mechanically impede hemorrhage without permitting inappropriate thrombosis.
On unactivated samples:
   i. Pipet 360 uL from red topped tube into cup, start TEG test
On activated samples:
   i. First, obtain the sample to be tested from lab. They should be weighed, bottled, oven activated (if needed), and capped prior to the start of the experiment. Inorganic solid samples are bottled in twice the amount that needs to be tested. For example, if channel two is to test 5 mg of inorganic solid A and blood, the amount weighed out in the bottle for channel two will be 10 mg. For 10 mg samples, 20 mg is weighed out, etc. See note below for reason.
   ii. For one activated run, 3 inorganic solid samples were tested at a time. An unactivated blood sample with no additive is run in the first channel. Channels 2, 3 and 4 are blood samples contacted with an inorganic solid.
   iii. Once ready to test, set one pipet to 720 uL and other pipet to 360 uL. Prepare three red capped tubes (plain polypropylene-lined tubes without added chemicals) to draw blood and prepare three red additional capped tubes to pour the inorganic solid sample into.
   iv. Draw blood from volunteer and bring back to TEG analyzer. Discard the first tube collected to minimize tissue factor contamination of blood samples. Blood samples were contacted with inorganic solid material and running in TEG machine prior to an elapsed time of 4-5 minutes from donor collection.
   v. Open bottle 1 and pour inorganic solid into red capped tube.
   vi. Immediately add 720 uL of blood to inorganic solid in tube.
   vii. Invert 5 times.
   viii. Pipet 360 uL of blood and inorganic solid mixture into cup.
   ix. Start TEG test.

Note: The proportions are doubled for the initial mixing of blood and inorganic solid because some volume of blood is lost to the sides of the vials, and some samples absorb blood. Using double the volume ensures that there is at least 360 uL of blood to pipet into cup. The proportion of inorganic solid to blood that we are looking at is usually 5 mg/360 uL, 10 mg/360 uL, and 30 mg/360 uL

The R(min) reported in the Tables below is the time from the start of the experiment to the initial formation of the blood clot as reported by the TEG analyzer. The TEG® analyzer has a sample cup that oscillates back and forth constantly at a set speed through an arc of 4° 45′. Each rotation lasts ten seconds. A whole blood sample of 360 ul is placed into the cup, and a stationary pin attached to a torsion wire is immersed into the blood. When the first fibrin forms, it begins to bind the cup and pin, causing the pin to oscillate in phase with the clot. The acceleration of the movement of the pin is a function of the kinetics of clot development. The torque of the rotating cup is transmitted to the immersed pin only after fibrin-platelet bonding has linked the cup and pin together. The strength of these fibrin-platelet bonds affects the magnitude of the pin motion, such that strong clots move the pin directly in phase with the cup motion. Thus, the magnitude of the output is directly related to the strength of the formed clot. As the clot retracts or lyses, these bonds are broken and the transfer of cup motion is diminished. The rotation movement of the pin is converted by a mechanical-electrical transducer to an electrical signal which can be monitored by a computer. The resulting hemostasis profile is a measure of the time it takes for the first fibrin strand to be formed, the kinetics of clot formation, the strength of the clot (in shear elasticity units of dyn/cm²) and dissolution of clot. The following data has been collected from a volunteer donor who had a suppressed coagulation system due to taking prescription aspirin, clopidogrel and warfarin. The unadulterated blood data is included with the data after addition of known amounts of materials.

| Material Added (5 mg) | R (min) | MA (mm) |
|---|---|---|
| CaZB 100 | 2.8 | 73.4 |
| Diafil 460 | 2.4 | 70.3 |
| Hi-Sil 250 | 2.2 | 65.7 |
| Control | 25.7 | 56.4 |

The materials studied include the following:
1. Calcium A zeolite—Ca-exchanged zeolite ZB-100, a NaA zeolite from UOP LLC, Des Plaines, Ill.
2. Diafil 460—World Minerals Inc. is headquartered in Santa Barbara, Calif. USA a high surface area ˜30 m²/g diatomaceous earth.
3. Hi-Sil 250—a precipitated silica (silica gel)—PPG Industries, Pittsburgh, Pa.

Highly significant clot acceleration was observed with the three materials. Other appropriate hemostatic or absorptive agents may also be added. These include but are not limited to chitosan and its derivatives, fibrinogen and its derivatives (represented herein as fibrin(ogen), e.g. fibrin, which is a cleavage product of fibrinogen, or super-absorbent polymers of many types, cellulose of many types, other cations such as calcium, silver, and sodium or anions, other ion exchange resins, and other synthetic or natural absorbent entities such as super-absorbent polymers with and without ionic or charge properties.

In addition, the inorganic solid may in addition have added to it vasoactive or other agents which promote vasoconstriction and hemostasis. Such agents might include catecholamines or vasoactive peptides. This may be especially helpful in its dry form so that when blood is absorbed, the additive agents become activated and are leached into the tissues to exert their effects. In addition, antibiotics and other agents which prevent infection (any bacteriocidal or bacteriostatic agent or compound) and anesthetics/analgesics may be added to enhance healing by preventing infection and reducing pain. In addition, fluorescent agents or components could be added to help during surgical removal of some forms of the mineral to ensure minimal retention of the mineral after definitive control of hemorrhage is obtained.

The formulations of the present invention may be administered to a site of bleeding by any of a variety of means that are well known to those of skill in the art. Examples include but are not limited to internally (e.g. by ingestion of a liquid or tablet form), directly to a wound, (e.g. by shaking powdered or granulated forms of the material directly into or onto a site of hemorrhage), by placing a material such as a bandage that is impregnated with the material into or onto a wound, by spraying it into or onto the wound, or otherwise coating the wound with the material. Bandages may also be of a type that, with application of pressure, bend and so conform to the shape of the wound site. Partially hydrated forms resembling mortar or other semisolid-semiliquid forms, etc. may be used to fill certain types of wounds. For intra-abdominal bleeding, we envision puncture of the peritoneum with a trocar followed by administration of inorganic solids of various suitable formulations.

Formulations may thus be in many forms such as bandages of varying shapes, sizes and degrees of flexibility and/or rigidity; gels; liquids; pastes; slurries; granules; powders; and other forms. The clay minerals can be incorporated into special carriers such as liposomes or other vehicles to assist in their delivery either topically, gastrointestinally, intracavitary (e.g., pleural, peritoneal, intracranial, intrauterine), or even intravascularly. In addition, combinations of these forms may also be used, for example, a bandage that combines a flexible, sponge-like or gel material that is placed directly onto a wound, and that has an outer protective backing of a somewhat rigid material that is easy to handle and manipulate, the outer layer providing mechanical protection to the wound after application. Both the inner and outer materials may contain clay minerals. Any means of administration may be used, so long as the mineral clay makes sufficient contact with the site of hemorrhage to promote hemostasis.

Compositions comprising clay minerals may be utilized to control bleeding in a large variety of settings, which include but are not limited to: (a) external bleeding from wounds (acute and chronic) through the use of liquids, slurries, gels, sprays, foams, hydrogels, powder, granules, or the coating of bandages with these preparations; (b) gastrointestinal bleeding through the use of an ingestible liquid, slurry, gel, foam, granules, or powder; (c) epistaxis through the use of an aerosolized powder, sprays, foam, patches, or coated tampon; (d) control of internal solid organ or boney injury through the use of liquids, slurries, sprays, powder, foams, gels, granules, or bandages coated with such; and (e) promotion of hemostasis, fluid absorption and inhibition of proteolytic enzymes to promote healing of all types of wound including the control of pain from such wounds. The inorganic material promotes clotting at a rate about 2-12 times faster than its absence. Blood clotting (as measured by TEG) is promoted generally in less than 10 minutes and preferably in less than 5 minutes.

Many applications of the present invention are based on the known problems of getting the surfaces of bandages to conform to all surfaces of a bleeding wound. The use of granules, powders, gels, foams, slurries, pastes, and liquids allow the preparations of the invention to cover all surfaces no matter how irregular they are. For example, a traumatic wound to the groin is very difficult to control by simple direct pressure or by the use of a simple flat bandage. However, treatment can be carried out by using an inorganic material in the form of, for example, a powder, granule preparation, gel, foam, or very viscous liquid preparation that can be poured, squirted or pumped into the wound, followed by application of pressure. One advantage of the preparations of the present invention is their ability to be applied to irregularly shaped wounds, and for sealing wound tracks, i.e. the path of an injurious agent such as a bullet, knife blade, etc.

1. A method for promoting blood clotting in a patient with a suppressed coagulation system comprising contacting a blood clot promoter to a bleeding site of a patient with a suppressed coagulation system wherein said blood clot promoter comprises an inorganic material selected from the group consisting of zeolitic and nonzeolitic materials, diatomaceous earth, glass powder or fibers, precipitated or fumed silica, kaolin and montmorillonite clays and calcium exchanged permutites.

2. The method of claim 1 wherein said suppressed coagulation system is a result of anticoagulant or platelet blocker therapy or both.

3. The method of claim 1 wherein said inorganic material is ion exchanged.

4. The method of claim 2 wherein said ion is calcium.

5. The method of claim 1 wherein said inorganic material is a diatomaceous earth.

6. The method of claim 1 wherein said inorganic material comprises non-mesoporous glass powder or fibers.

7. The method of claim 1 wherein said inorganic material comprises calcium polyphosphate glass.

8. The method of claim 1 wherein said inorganic material comprises silica gel.

9. The method of claim 1 wherein said inorganic material comprises precipitated or fumed silica.

10. The method of claim 1 wherein said blood clot promoter is contained within a porous carrier selected from the group consisting of woven fibrous articles, non-woven fibrous articles, puff, sponges and mixtures thereof.

11. The method of claim 1 further comprising the step of removing all or a portion of said inorganic material from a wound.

12. The method of claim 1 wherein said inorganic material is in the form of a free flowing powder.

13. The method of claim 1 wherein said inorganic material promotes blood clotting at a rate about 2-12 times faster than in its absence.

14. The method of claim 1 wherein said inorganic material promotes blood clotting in less than about 10 minutes.

15. The method of claim 1 wherein said inorganic material promotes blood clotting in less than about 5 minutes.

16. The method of claim 1 wherein said blood clot promoter further comprises antibiotics, antifungal agents, antimicrobial agents, anti-inflammatory agents, analgesics, bacteriostatics, compounds containing silver ions, chitosan, fibrin(ogen), thrombin, superabsorbent polymers, calcium, polyethylene glycol, dextran, vasoactive catecholamines, vasoactive peptides, electrostatic agents, anesthetic agents or fluorescent agents.

17. The method of claim 1 wherein said bleeding site is an external wound.

18. The method of claim 1 wherein said bleeding site is an internal wound.

19. The method of claim 1 wherein said blood clotting promoter is applied topically, gastrointestinally, intracavitary or intravascularly.
